(19) United States
(12) Patent Application Publication
(10) Pub. No.: US 2008/0300545 A1
(43) Pub. Date: Dec. 4, 2008

(54) Trocar Cannula with an Elastic Ring

(76) Inventor: Chin-Cheng Hsieh, Hsin Tien City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/780,545

(22) Filed: Jul. 20, 2007

(30) Foreign Application Priority Data
Jun. 1, 2007 (TW) 096209127

(51) Int. Cl. A61B 17/34

(52) U.S. Cl. 604/167.03; 606/185

(57) ABSTRACT

This invention provides a trocar cannula adapted to form a seal around a surgical instrument, the trocar cannula comprising: a housing for receiving the surgical instrument; an elastomeric septum seal disposed in the housing, the elastomeric septum seal comprising a septum seal defining an orifice having a diameter substantially no greater than the diameter of the surgical instrument so that during insertion of the surgical instrument, the septum seal forms a seal with the surgical instrument; a cannula tube connected to the housing; a zero closure valve disposed between the cannula tube and the housing; and an elastic ring disposed surrounding the elastomeric septum seal, the elastic ring being capable of changing its shape in accordance with the movement of the elastomeric septum seal so as to enable the septum seal to maintain a tight seal around the surgical instrument when the surgical instrument is moved.

### BACKGROUND OF THE INVENTION

1. Field of the Invention

The present invention relates to a trocar cannula; more specifically, the present invention relates to a trocar cannula with an elastic ring.

2. Description of the Related Art

Laparoscopic surgery is a popular operation technique used extensively in recent years. During the operation, operators usually cut open a patient's abdomen with a trocar cannula and an obturator and then inflate the abdomen with a gas such as carbon dioxide through the trocar cannula to increase the operable space therein. In addition, the trocar cannula may also be used as the passage for various surgical instruments and maintain the abdominal pressure within a specific range.

In general, most trocar cannulas have an anti-leakage structure capable of preventing gas from escaping from a patient's abdomen; the anti-leakage structure may create a tight seal around the surgical instruments so that the patient's abdominal pressure may be maintained within the specific range even when the surgical instruments are moved. Today, almost all trocar cannulas adapt an elastomeric septum seal to achieve the aforementioned anti-leakage effect. At the center of the elastomeric septum seal, an orifice having a diameter that is less than those of the surgical instruments is provided; due to its inherent flexibility, the orifice is capable of forming a seal with the surgical instrument when it is penetrated by the surgical instrument, helping to overcome the gas leakage problem that results from frequent insertion and withdrawal of the surgical instruments.

However, when a surgical instrument is moved horizontally, the orifice, due to the fixed structure of the elastomeric septum seal, cannot be moved along with the surgical instrument. The resulting deformation of the orifice may cause gas leakage.

In prior arts, several inventions have been proposed to obviate the leakage problem mentioned above. For example, in U.S. Pat. No. 5,578,016, a stopcock, which acts as an eyeball-shaped elastomeric septum seal, is disclosed; the stopcock has a hollow passage perforated through a ball structure. When a surgical instrument is inserted through the elastomeric septum seal, it is tightly sealed; in addition, the eyeball-shaped elastomeric septum seal may rotate without deformation to maintain a tight seal around the surgical instrument when the surgical instrument is moved. Moreover, U.S. Pat. No. 5,385,553 discloses a trocar with a floating septum seal; a flexible valve disposed in the channel includes first portions defining an orifice through the valve and second portions disposed outwardly of the first portions. The second portions are provided with a greater flexibility so that when the instrument is inserted off-axis, the second portions of the valve will deform before the first portions, allowing the orifice to move without substantial deformation to the off-axis location.

### SUMMARY OF THE INVENTION

It is therefore an objective of the present invention to provide a trocar cannula having a movable elastomeric septum seal; when a surgical instrument inserted therethrough is moved horizontally, the elastomeric septum seal may be moved along with the surgical instrument; thus, the orifice on the elastomeric septum seal may not be deformed and is capable of maintaining a tight seal around the surgical instrument.

It is another objective of the present invention to provide a trocar cannula having an elastomeric septum seal with an elastic ring. When the elastomeric septum seal is moved by a surgical instrument, the elastic ring disposed surrounding the elastomeric septum seal may deform correspondingly; when the surgical instrument is retracted out of the elastomeric septum seal, the septum seal may recover from the deformed state and, furthermore, maintain the elastomeric septum seal in its original state and return the orifice in the elastomeric septum seal to the center, largely facilitating the follow-up insertion of surgical instruments.

It is still another objective of the present invention to provide a trocar cannula having a funnel-shaped septum seal for directing a surgical instrument to the orifice thereon so as to facilitate the insertion of the surgical instrument.

It is also an objective of the present invention to provide a trocar cannula having a protection member mounted on a septum seal; the protection member is adapted to protect the septum seal and prevent it from being pierced or damaged by a surgical instrument. In addition, in the situation where the septum seal has a non-planar structure, the protection member may also prevent inversion of the septum seal upon withdrawal of the surgical instrument.

Certain of the foregoing and related objectives are readily obtained according to the present invention in a trocar cannula adapted to form a seal around a surgical instrument, the trocar cannula comprising: a housing for receiving the surgical instrument; an elastomeric septum seal disposed in the housing, the elastomeric septum seal comprising a septum seal defining an orifice having a diameter substantially no greater than the diameter of the surgical instrument so that during insertion of the surgical instrument, the septum seal forms a seal with the surgical instrument; a cannula tube connected to the housing; a zero closure valve disposed between the cannula tube and the housing; and an elastic ring disposed surrounding the elastomeric septum seal, the elastic ring being capable of changing its shape in correspondence to the movement of the elastomeric septum seal so as to enable the septum seal to tightly fasten the surgical instrument when the surgical instrument is moved.

Moreover, the elastomeric septum seal may further comprise at least one protection member mounted on the septum seal and a pair of fitting members for securing the relative positions of the protection member and the septum seal. The protection member is adapted to protect the septum seal and prevent it from being pierced or damaged by a surgical instrument. In addition, in the situation where the septum seal has a non-planar structure, the protection member may also prevent inversion of the septum seal upon withdrawal of the surgical instrument. The fitting members may be two plastic rings for securing the protection member and the septum seal by sandwiching the protection member and the septum seal therebetween.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustrations only, and not as a definition of the invention.

In the drawings, wherein similar reference numerals denote similar elements throughout the several views:

FIG. 1 is an illustrative diagram of the trocar cannula of the present invention.

FIG. 2 illustrates an exploded view of an embodiment of the trocar cannula of the present invention.

FIG. 3 illustrates a sectional view of the trocar cannula of the present invention.

FIG. 4 illustrates a sectional view of the trocar cannula of the present invention in use.

### DETAILED DESCRIPTION OF THE PREFERRED EMBODIMENT

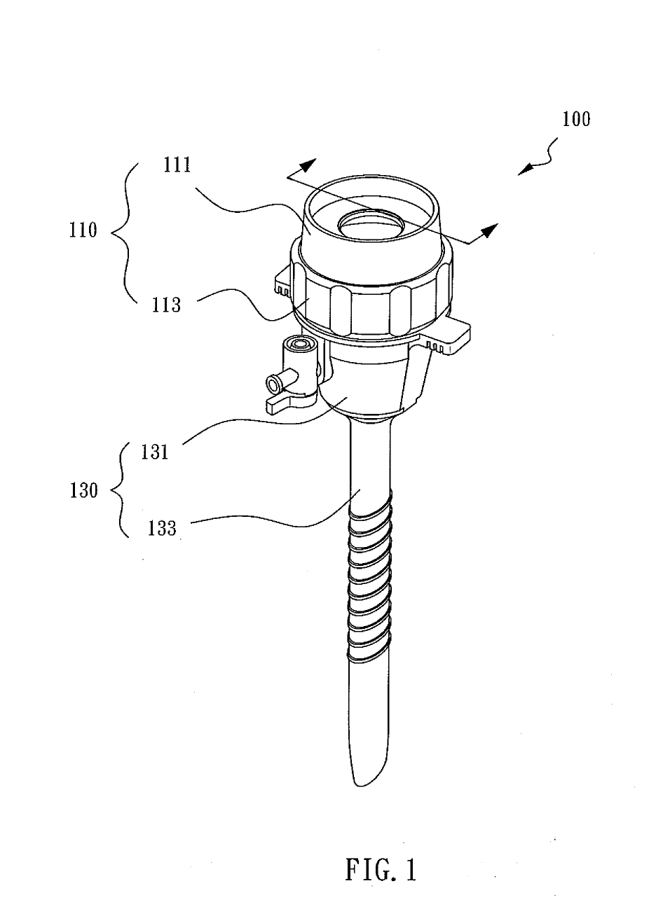

Please refer to FIG. 1 for an illustrative diagram of the trocar cannula 100 of the present invention. The trocar cannula 100 mainly comprises two major parts—a housing 110, in which an elastomeric septum seal is disposed, for receiving a surgical instrument, and a cannula tube 130 connected to the housing 110. The housing 110 has a penetrable structure, and in this embodiment the housing 110 consists of an upper cover 111 and a lower cover 113 connected mutually by buckling means; the upper cover 111 and the lower cover 113 may define an accommodation space in which the elastomeric septum seal is disposed. However, it should be noted that the shape and structure of the housing 110 is not limited to what is disclosed in this embodiment; for example, the housing may also be assembled by other means.

In this embodiment, the cannula tube 130 comprises a connection part 131 and a tubular part 133 extended from the connection part 131. The upper portion of the connection part 131 may be connected to the lower cover 113 of the housing 110 in a detachable manner. One end of the tubular part 133 is connected to the connection part 131, and the other end has a pointed slant for penetrating a patient's abdomen. In some situations, the tubular part 133 and the connection part 131 may be combined detachably; however, depending on the kind of mold used, they may also be fabricated as a one-piece component.

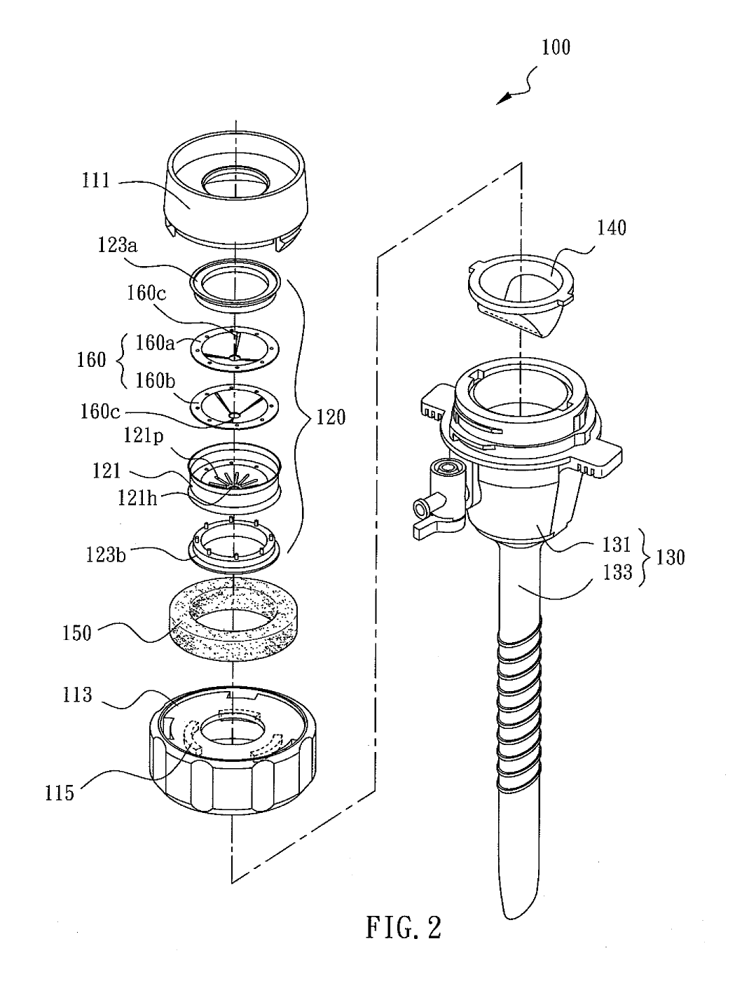

Refer to FIG. 2 for an exploded view of an embodiment of the trocar cannula of the present invention. As shown in FIG. 2, the trocar cannula 100 comprises an elastomeric septum seal 120 which has a septum seal 121, the septum seal 121 defining an orifice 121h having a diameter substantially no greater than the diameter of the surgical instrument so that during insertion of the surgical instrument the septum seal 121 forms a seal with the surgical instrument.

In this embodiment, septum seal 121 has a funnel-shaped structure protruding toward the cannula tube 130. When a medical operator inserts a surgical instrument into the trocar cannula 100, the septum seal 121 may provide a direction effect by its funnel-shaped structure, largely simplifying the use of the trocar cannula 100 and facilitating the operation process. In addition, the septum seal 121 has embossment 121p for reducing the friction between the septum seal and the surgical instrument.

Furthermore, in order to prevent the septum seal 121 from being pierced or damaged during the surgical operation, the elastomeric septum seal 120 may further comprise a protection member 160 secured on the septum seal 121 to provide necessary protection. In some situations where the septum seal 121 has a non-planar structure, such as the funnel-shaped structure mentioned above, the protection member 160 may also prevent inversion of the septum seal 121 upon withdrawal of the surgical instrument. In this embodiment, the elastomeric septum seal 120 comprises two protection members 160a and 160b, both of which have a slit 160c for providing the protection member 160 with a tensile effect. The two protection members 160a and 160b may be stacked in a staggered manner to prevent a surgical instrument from piercing the septum seal 121 through the slit 160c. Also, in this embodiment the protection member 160 may be without limitation a common plastic disc made of, for example, polyethylene or polypropylene. In addition, the protection member 160 may also be a fabric or other similar fibers covering the septum seal 121.

In this embodiment, a pair of fitting members 123a and 123b are adapted to secure the relative positions of the protection member 160 and the septum seal 121; the pair of the fitting members 123a and 123b may sandwich the protection member 160 and the septum seal 121 therebetween. Because several apertures may be formed on the protection member 160 and the septum seal 121, the fitting members 123a and 123b may secure the protection member 160 and the septum seal 121 through the apertures.

More importantly, the present invention provides an elastic ring 150 disposed surrounding the elastomeric septum seal 120. The two components may be disposed in the accommodation space defined by the upper cover 111 and the lower cover 113. In addition, a zero closure valve 140 is disposed in the connection part 131 of the cannula tube 130; the zero closure valve 140 may be, but is not limited to, a duckbill valve or a cross valve. It should be noted that, in this embodiment, the lower cover 113 has at least one protrusion 115 for securing the zero closure valve 140 in the connection part 131.

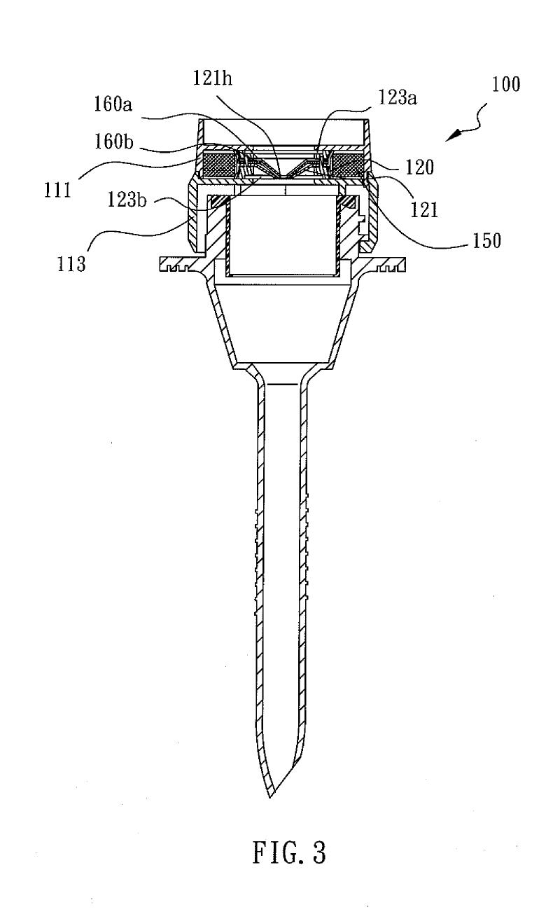

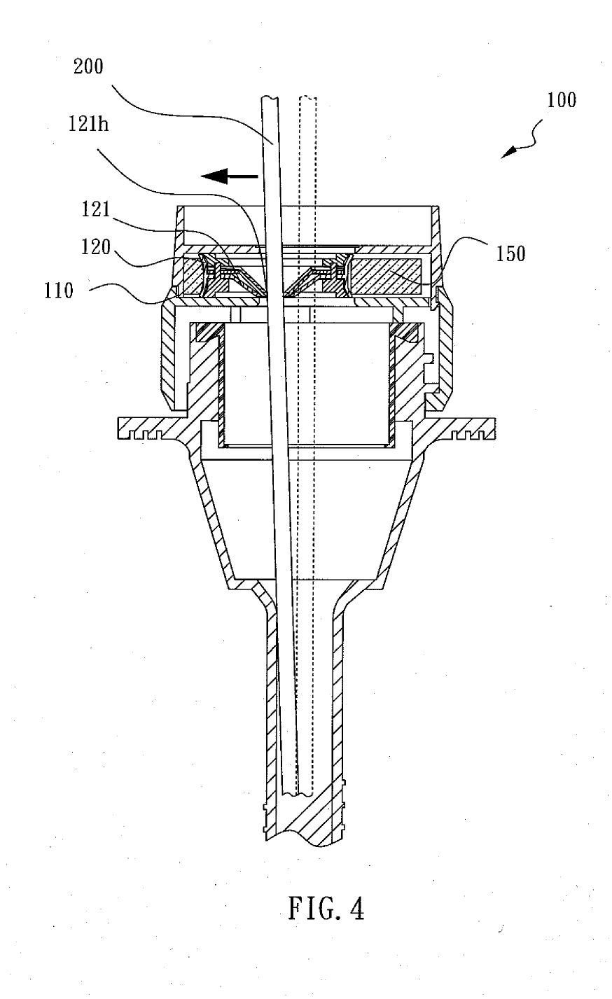

Refer to FIG. 3 for a sectional view of the trocar cannula 100 of the present invention. In this embodiment, the elastomeric septum seal 120 comprises a septum seal 121, two protection members 160a and 160b, and a pair of fitting members 123a and 123b; the protection members 160a and 160b are stacked on the septum seal 121 in a staggered manner, and the fitting members 123a and 123b secure the septum seal 121 and the protection members 160a and 160b therebetween. The elastomeric septum seal 120 is disposed within a space defined by the upper cover 111 and the lower cover 113, and an elastic ring 150 is provided surrounding the elastomeric septum seal 120. The elastic ring 150 may be deformed when the elastomeric septum seal 120 is moved; thus, the orifice 121h on the elastomeric septum seal 120 may consistently fasten a surgical instrument without deformation during the laparoscopic surgical operation, whether the surgical instrument is being moved or not. Also, the orifice 121h may not be pulled by the surgical instrument into a “cat-eye” shape, such that degradation of the seal around the surgical instrument can be prevented. Please refer to FIG. 4 for a sectional view of the trocar cannula 100 of the present invention. In use, a surgical instrument 200 is inserted through housing 110 and the elastomeric septum seal 120 disposed therein. Since the diameter of the orifice 121h on the septum seal 121 is no greater than that of the surgical instrument 200, the septum seal 121, by its flexibility, is capable of forming a seal with the surgical instrument 200. When the surgical instrument 200 is moved in a direction parallel to the elastomeric septum seal 120, the elastomeric septum seal 120 may be moved correspondingly due to the fact that the elastomeric septum seal 120 is actively installed. In addition, the elastomeric septum seal 120 is surrounded by the elastic ring 150, which may be deformed when the elastomeric septum seal 120 is moved. In a preferred embodiment, the elastic ring 150 may be a sponge or its equivalent, which, due to its inherent loose texture and compressible property, may not cause too much drag when the surgical instrument 200 is moved. When a medical operator needs to change the surgical instrument 200 during the operation, the elastic ring 150 may recover from the deformed state to its original shape by the intrinsic flexibility once the surgical instrument 200 is withdrawn from the elastomeric septum seal 120. In the meantime, the elastomeric septum seal 120 may be returned to its original position. Accordingly, medical operators will not have to spend too much time feeding the surgical instrument 200 into the orifice 121h on the septum seal 121.

It will be understood that many other modifications can be made to the various disclosed embodiments without departing from the spirit and scope of the invention. For these reasons, the above description should not be construed as limiting the invention, but should be interpreted as merely exemplary of preferred embodiments.

What is claimed is:

1. A trocar cannula adapted to form a seal around a surgical instrument, the trocar cannula comprising:
a housing for receiving the surgical instrument;
an elastomeric septum seal disposed in the housing, the elastomeric septum seal comprising a septum seal defining an orifice having a diameter substantially no greater than the diameter of the surgical instrument so that during insertion of the surgical instrument the septum seal forms a seal with the surgical instrument;
a cannula tube connected to the housing;
a zero closure valve disposed between the cannula tube and the housing; and
an elastic ring disposed surrounding the elastomeric septum seal, the elastic ring being capable of changing its shape in accordance with the movement of the elastomeric septum seal so as to enable the septum seal to tightly fasten the surgical instrument when the surgical instrument is moved.

2. The trocar cannula as claimed in claim 1, wherein the elastic ring is a sponge.

3. The trocar cannula as claimed in claim 1, wherein the housing comprises an upper cover and a lower cover mutually connected by buckling means.

4. The trocar cannula as claimed in claim 1, wherein the cannula tube comprises a connection part and a tubular part, the zero closure valve being disposed in the connection part.

5. The trocar cannula as claimed in claim 4, wherein the housing comprises at least one protrusion for securing the zero closure valve in the connection part.

6. The trocar cannula as claimed in claim 1, wherein the septum seal protrudes toward the cannula tube.

7. The trocar cannula as claimed in claim 6, wherein the septum seal has embossment capable of reducing the friction between the septum seal and the surgical instrument.

8. The trocar cannula as claimed in claim 1, further comprising a protection member mounted on the septum seal.

9. The trocar cannula as claimed in claim 8, wherein the protection member has a slit.

10. The trocar cannula as claimed in claim 9, comprising two protection members arranged in a staggered manner so that the slits are offset from one another.

11. The trocar cannula as claimed in claim 1, wherein the elastomeric septum seal further comprises:
at least one protection member mounted on the septum seal; and
a pair of fitting members for securing the relative positions of the protection member and the septum seal.

12. The trocar cannula as claimed in claim 11, wherein the protection member is a made of plastic.

13. The trocar cannula as claimed in claim 11, wherein the pair of fitting members are two plastic rings.

14. The trocar cannula as claimed in claim 1, wherein the housing is detachably connected to the cannula tube.
